(19) United States
(12) Patent Application Publication
(10) Pub. No.: US 2026/0191450 A1
(43) Pub. Date: Jul. 9, 2026

(54) MOBILE DEVICE BASED HANG GRIP STRENGTH MEASUREMENT

(71) Applicant: The Regents of the University of California, Oakland, CA (US)

(72) Inventors: Colin Barry, La Jolla, CA (US); Edward Jay Wang, La Jolla, CA (US)

(21) Appl. No.: 19/134,112

(22) PCT Filed: Nov. 28, 2023
(86) PCT No.: PCT/US2023/081474

§ 371 Date: May 29, 2025

Related U.S. Application Data
(60) Provisional application No. 63/385,379, filed on Nov. 29, 2022.

(51) Int. Cl. A61B 5/22; A61B 5/00

(52) U.S. Cl. CPC A61B 5/225; A61B 5/0051; A61B 5/7264; A61B 5/742; A61B 5/746

(57) ABSTRACT

Disclosed are systems, devices and methods for mobile device based hand grip strength measurement. In some aspects, a mobile device includes a vibration motor, an inertial motion unit (IMU), a processor, and non-transitory computer-readable memory, in which the processor is configured to perform operations including: causing the vibration motor to vibrate; acquiring, using the inertial measurement unit of the mobile device, IMU data indicative of vibration damping that is caused by a user applying a force on the mobile device; and determining the applied force based on a force model and the IMU data.

### CROSS-REFERENCE TO RELATED APPLICATIONS

This patent document claims priority to and benefits of U.S. Provisional Patent Application No. 63/385,379 entitled “SMARTPHONE-BASED HAND GRIP STRENGTH MEASUREMENT” filed on Nov. 29, 2022. The entire content of the aforementioned patent application is incorporated by reference as part of the disclosure of this patent document.

### TECHNICAL FIELD

This patent document relates to mobile device based hand grip strength measurement.

### BACKGROUND

Hand grip strength (HGS) is a functional biomarker for overall status of a user that may be used in an individual test or in a battery of tests. As a measure of physical force generation, HGS may provide insight into muscle mass decline or bone density issues in the user. Also, there may exist correlations between HGS and cognitive functioning. These correlations may be more than simple correlations between aging related decline in muscle mass and cognitive systems. Nervous system declines may impact motor system capacity and dexterity separate from a musculoskeletal decline. Thus, conditions affecting neurological decline may also impact the nervous system and

HGS. In this way, the HGS measurement may provide a metric for fragility.

### SUMMARY

The present document discloses methods, devices, and systems for hand grip strength measurement using sensor data acquired by built-in sensors of mobile devices with no additional attachments.

An aspect of the present document relates to a mobile device configured to measure hand grip strength using sensor data acquired by built-in sensors of the mobile device without attachments. The mobile device may include: a vibration motor, an inertial motion unit (IMU), a processor, and non-transitory computer-readable memory that stores instructions that, when executed by the processor, cause the processor to perform operations including: causing the vibration motor to vibrate; acquiring, using the inertial measurement unit of the mobile device, IMU data indicative of vibration damping that is caused by a user applying a force on the mobile device; determining the applied force based on a force model and the IMU data.

An aspect of the present document relates to a method for measuring hand grip strength using sensor data acquired by built-in sensors of the mobile device without attachments. The method may include: causing a vibration motor of the mobile device to vibrate; acquiring, using an inertial measurement unit (IMU) of the mobile device, IMU data indicative of vibration damping that is caused by a user applying a force on the mobile device; determining the applied force based on a force model and the IMU data.

A further aspect of the present document relates to one or more non-transitory computer-readable media storing instructions that, when executed by one or more processors of a mobile device, cause the mobile device to perform any one or more of the solutions described herein.

The above and other aspects of the present document and their implementations and applications are described in greater detail in the drawings, the description, and the claims.

### BRIEF DESCRIPTION OF THE DRAWINGS

FIG. 1 illustrates a hand grip strength measurement using a hand dynamometer.

FIG. 2 shows a diagram of an example system implementing the disclosed technology in accordance with the disclosed technology.

FIG. 3 illustrates an exemplary block diagram of the various components of a mobile device in accordance with some embodiments of the present document.

FIG. 4 illustrates a flowchart of a process for a mobile device based hand grip strength measurement in accordance with some embodiments of the present document.

FIG. 5 illustrates regression and Bland Altman plots for force measurements of three different smartphones in accordance with some embodiments of the present document.

FIG. 6 illustrates a comparison between a force measured using a force sensor with a corresponding force determined based on a force model in accordance with some embodiments of the present document.

FIG. 7 illustrates results of an HGS measurement using a mobile device categorized as levels in accordance with some embodiments of the present document.

FIG. 8 illustrates an example graphic user interface of an application for HGS measurement in accordance with some embodiments of the present document.

### DETAILED DESCRIPTION

The present document describes systems, devices, and methods for measuring hand grip strength using built-in sensors of a mobile device. This methodology is applicable to any mobile device with a vibration motor and an inertial measurement unit (IMU). For the mobile phone application disclosed herein, a mobile device may be caused to vibrate driven by its internal vibration motor, and monitor the damping of these vibrations resulting from a force a user applies using the mobile device's IMU, and quantify the applied force based on a force model and the IMU data. The mobile device may determine a hand grip strength of the user based on the IMU data acquired using the built-in sensors already in the mobile device, needs no hardware modifications, no physical attachments, and can be enabled solely with software by downloading an application on any mobile device, as discussed in further detail below. Accordingly, the technology may provide large scale screening opportunities for mental or physical impairment. As a software enabled measurement for any mobile device with an IMU and a vibration motor, this technology can be quickly proliferated, including into underserved communities.

Since cognitive and musculoskeletal deterioration may impact HGS, HGS may be used as a functional biomarker to provide health professionals with warning signs for patients experiencing significant mental or physical decline. As an individual ages, there may be an expected muscle mass loss and decline in HGS; however, significant and unexpected declines in HGS may likely be representative of a mental or physical impairment that may continue to worsen health if left untreated.

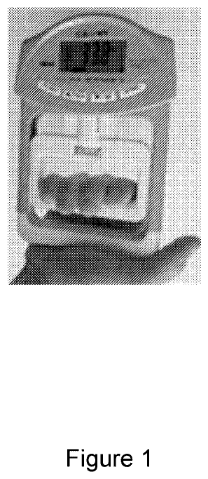

HGS may be measured with a hand dynamometer as illustrated in FIG. 1, a device with a strain gauge sensor. A hand dynamometer may generally measure the maximum 5-second isometric strain. For example, a user may perform an HGS test using a single hand with the metacarpal on the bottom of the device and the pull bar between the first and second finger joint. The user may pull with maximum effort using a measuring hand and receive a strength classification (weak, medium, or strong) based on the measurement and demographic.

The act of measuring the force exerted by a user's hand using a dynamometer may be similarly performed with built-in sensors and actuators of a commodity mobile device. While a mobile device vibrates, the IMU (including an accelerometer and/or a gyroscope) readings may reflect the vibration. These signals may be dampened by adding force (or weight). Instead of focusing on static or binary metrics, HGS may be measured using the dampening of the IMU (including, e.g., accelerometer and gyroscope) of the mobile device during vibration. As illustrated elsewhere in the present document, HGS sensing on the mobile device may provide a result on a continuous scale and/or categorized as levels.

The mobile device based HGS measurement as disclosed herein needs no attachment and functions on mobile devices of various types with a vibration motor and an IMU. Most mobile devices already contain an IMU with an accelerometer and gyroscope for other purposes including, e.g., to measure the linear acceleration and angular velocity that enables position inferences by such mobile devices widely used for rotating the screen, playing games, counting steps, gesture recognition, and much more. Similarly, most mobile devices already contain a vibration motor to provide haptic feedback for messages, phone calls, and screen interactions, etc. Such a mobile device may implement an HGS measurement based on a force damping technique that utilizes only the vibration motor and IMU of the mobile device. To measure force, the mobile device may be set to vibrate (e.g., maximally vibrate) while the IMU measures the motion as the device oscillates (from the vibration). When force is applied during vibration, the IMU signal changes as the oscillations are altered or dampened. According to embodiments of the present documents, the vibration dampening is measured and relied upon to model the applied force as a damping force on the vibration. By relying on components common to mobile devices of different types, the HGS measurement as disclosed herein is functional across mobile devices of different types (e.g., a type may correspond to a model by a manufacturer).

According to some embodiments of the present document, IMU data that includes multi-axis accelerometer data and multi-axis gyroscope data collectively representing a vibration damping is used to determine a corresponding force that causes the vibration damping. This method recognizes a coupling between different axes of gyroscope and accelerometer data that respond differently to an applied force. Due to the conservation of energy, an applied force does not solely cause vibration damping or decreasing in amplitude along all IMU axes. Instead, some axes may even increase as the applied force to damp the vibration motion in one axis may cause the vibration energy to be transmitted into another axis. The disclosed method takes multiple inputs from both the accelerometer and gyroscope as inputs, investigating frequency, amplitude, and relationships between different axes. As used herein, “damping” or “vibration damping” refers to recorded alterations in the device's motion that result from the applied hand grip force.

An applied force causing vibration damping of the mobile device may be quantified based on a force model trained to correlate (1) IMU data measured by the mobile device that is indicative of vibration damping of the mobile device with (2) forces that are applied to the mobile device and cause the corresponding vibration damping. The force model may include a machine learning algorithm, e.g., a multivariate linear regression algorithm. The correlation may depend on the configuration of the mobile device. Damping of vibration may depend on one or more factors that contribute to how the mobile device absorbs and dissipates vibrational energy. Examples of such factors include the strength of the vibration, the load distribution within the mobile device (including, e.g., how the weight and components are distributed within the mobile device), materials used in the mobile device (including, e.g., density, elasticity, and internal friction), the overall design (including, e.g., shape and structural integrity, features such as ribs, gussets), interaction between components within the device, or the like, or a combination thereof. Moreover, the damping as measured by the IMU of the mobile device may depend on one or more factors including, e.g., the position of the IMU relative to the vibration motor(s), sensor parameters including, e.g., sensitivity, resolution, noise level, or the like, or a combination thereof. The technology disclosed herein is versatile and translatable across mobile devices of various types such that similar force estimation performance may be obtained across various mobile devices. A force model may be determined for a type of mobile devices (e.g., a model by a manufacturer) and used to calibrate mobile devices of the same type.

The calibration step may allow a standardized measurement across mobile devices. The calibration may ensure that different mobile devices of a same type or different types are configured to measure force consistently. This is beneficial because the developing the force model is an automated or at least partially automated process that can be completed within a short period of time (e.g., a day) and does not take large participant recruitment or clinical measurements.

Technical improvements offered in the embodiments disclosed herein include the methods for performing HGS measurements that can be applied across different types (e.g., models) of mobile devices without hardware modifications or attachments and instead can be achieved by downloadable applications. Generalizing sensor-based measurements across the large variation of mobile devices is an established issue that significantly limits the impact of work in the mobile health space. The cross-device compatibility study result disclosed herein is evidence that the force damping technique as disclosed allows the HGS measurement to be functional on a range of mobile devices. The data processing and modeling demonstrate that many mobile devices of different types are capable to perform HGS measurements after a mobile device type based calibration. With tens of millions of users for mobile devices of each one of popular types, a single factory calibration may enable millions of mobile devices to perform hand grip strength measurements.

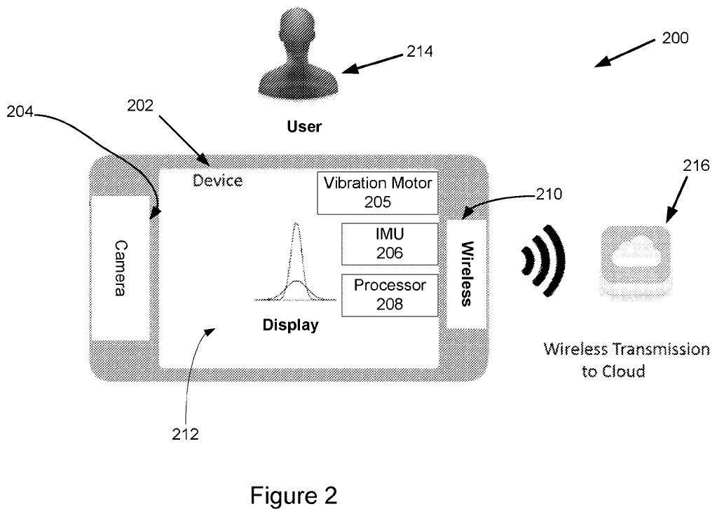

FIG. 2 shows an exemplary system implementing the disclosed technology of mobile device based hand grip strength measurement. The system 200 includes a mobile device 202 that can include at least one of a camera 204, a vibration motor 205, an IMU 206, a processor 208, a wireless transmitter 210, and a display 212. The processor 208 can control operations of the mobile device 202 (e.g., causing the vibration motor 205 to vibrate, causing the camera 204 and/or the IMU 206 to acquire data), receive and process sensor data (e.g., image data acquired by the camera 204, IMU data acquired by the IMU 206), and run algorithms (e.g., a force model) on the sensor data and generate results (e.g., an applied force to bring about vibration damping, a force plot, diagnosis based on the determined HGS, etc.). As shown in FIG. 2, the mobile device 202 may communicate with a user 214 or an external device or system (e.g., a cloud server 216). For example, the mobile device 202 can send a report of the state of the system to a cloud server 216 using, for example, a wireless transmitter 210. As another example, the mobile device 202 may communicate with the user 214 via the display 212. The display 212 may be a touch screen configured as a graphical user interface such that the mobile device 202 may present data or results to the user 214 via the display 212 and receive user input via the display 212. In some embodiments, the mobile device 202 may be a smartphone, a tablet, etc.

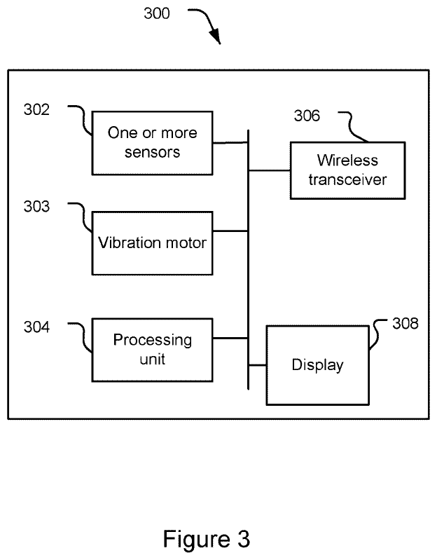

FIG. 3 illustrates an exemplary block diagram of the various components of a mobile device in accordance with some embodiments of the present document. The mobile device 300 is an example of the mobile device 202 as illustrated in FIG. 2. In some embodiments, the mobile device 300 may be a smartphone, a tablet, etc. In some embodiments, a mobile device 300 can vibrate driven by a built-in vibration motor 303, measure, using IMU, vibration damping caused by a user applying a force on the mobile device 300, and estimate HGS using a force model based on the IMU data. The mobile device 300 includes one or more sensors 302 that can gather data, a processing unit 304 connected to the one or more sensors 302 and capable of executing a force model on the gathered data, a wireless transceiver 306 connected to the processing unit 304, and a display 308 connected to the processing unit 304. The one or more sensors 302 may include one or more of a camara or an IMU. An IMU of the mobile device 300 may include an accelerometer and a gyroscope.

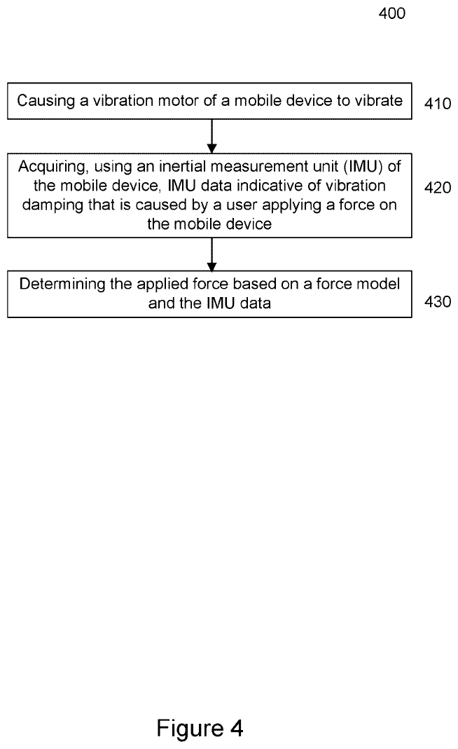

FIG. 4 illustrates a flowchart of a process for a mobile device based hand grip strength measurement in accordance with some embodiments of the present document. The process 400 may be implemented on a mobile device (e.g., the mobile device 202, the mobile device 300).

At block 410, the process 400 includes causing a vibration motor of the mobile device to vibrate. The vibration motor may be one already built-in in the mobile device. For example, the vibration may be driven by the vibration motor that is used to provide haptic feedback for messages, phone calls, and screen interactions, etc. The vibration motor may be caused to vibrate at a specific level (e.g., at a maximal level) of the vibration motor.

At block 420, the process 400 includes acquiring, using an inertial measurement unit (IMU) of the mobile device, IMU data indicative of vibration damping that is caused by a user applying a force on the mobile device. To measure HGS, the user may apply the force by gripping the mobile device with a hand.

In some embodiments, the process 400 may include providing a visual guide to be presented on a display of the mobile device. The visual guide (also referred to as a visual signifier) includes indicia to guide the user in applying the force by gripping the mobile device. The process 400 may include providing information on how a user performs an HGS measurement using the mobile device including, e.g., how a user should sit, apply a force, or the like, or a combination thereof, as part of the preparation. Merely by way of example, the instructions include that the user should sit upright with the measurement device (the mobile device) held directly out in front of the user in a neutral wrist position with a 90 degree elbow bend, that the user's feet should be flat on the ground, that the user should remain calm and breathe normally during the measurement, that the user should be gripping the mobile device with edges of the mobile device resting between the fingers and the palm of a hand of the user. The mobile device may provide such instructions to the user via a user interface implemented on, e.g., a display of the mobile device. The instructions may be presented in the form of text, an image, a cartoon, a video, an audio message, or the like, or a combination thereof. For example, the instructions may be presented as a visual guide including an image or a cartoon showing suggested fingers of a user's and/or suggested positions of fingers of a user's hand with which the user grips the mobile device. As another example, the instructions may be presented as a video showing a model user performs an HGS measurement using a sample mobile device.

Following the instructions, the user may apply a damping force by gripping the mobile device when the mobile device vibrates. The user may apply a maximal force for a time period (e.g., approximately 5 seconds) until the measurement is complete. The mobile device may inform the user when the measurement is complete.

In some embodiments, the process 400 may include receiving information regarding an execution of an HGS measurement by the user and evaluate whether the user is ready to perform a measurement and/or whether a measurement is valid. For example, the mobile device may detect the positions of the user's fingers that grip the mobile device and evaluate whether the gripping is appropriate for the user to proceed with the measurement. In some embodiments, the position detection may be performed based on one or more images captured by a camera of the mobile device or another device (e.g., another mobile device, a camera mounted on a wall or placed on a surface, etc.). In some embodiments, the mobile device includes a touch screen (e.g., the display 212) configured to sense contact by the user, and the process 400 may detect contact of one or more fingers of the user with the touch screen and determine the position(s) of the one or more fingers based on the detected contact. As a further example, the mobile device may detect the posture of the user, or a portion thereof (e.g., the posture of an arm or wrist of the user) by capturing one or more images of the user, or a portion thereof, to assess whether the user is properly positioned to perform a measurement. In some embodiments, the process 400 may include providing a notification to the user based on the evaluation. For example, in response to an evaluation concluding that the user needs to make an adjustment (e.g., an adjustment in terms of gripping or arm posture), the mobile device can provide a notification to the user to suggest the adjustment and/or make further detection or evaluation as to whether the user has made the suggested adjustment.

The IMU of the mobile device may include a multi-axis accelerator and a multi-axis gyroscope. The vibration motor may be one already built-in in the mobile device for other purposes including, e.g., to measure the linear acceleration and angular velocity that enables position inferences by such mobile devices widely used for rotating the screen, playing games, counting steps, gesture recognition, etc.

The IMU data may include multi-axis accelerometer data and multi-axis gyroscope data. For example, the IMU data may include three-axis accelerometer data and three-axis gyroscope data. The three-axis accelerometer data may include linear accelerations of the IMU in various axes (indicative of the smartphone motion). Depending on how energy dissipates during vibration damping, linear accelerations of the IMU may change in different patterns. For example, the linear accelerations along different directions may decrease at different rates or even following opposite trends (e.g., the linear acceleration in one direction increases, while the linear acceleration in one direction decreases) in some portion(s) of the period the damping force is applied. Similarly, the angular velocities of the IMU along different directions (indicative of the smartphone motion) may change in different patterns in response to the damping force the user applies. Accordingly, the IMU data including the multi-axis accelerometer data and the multi-axis gyroscope data may be used in combination to provide a comprehensive representation of the vibration damping and therefore improve the accuracy of the determined applied force.

Each axis of the multi-axis accelerometer data and the multi-axis gyroscope data may include a time-series signal. A signal of an axis of the IMU data may contain high frequency components from the vibration motor and low frequency components corresponding to noise. In some embodiments, the raw IMU data may be sampled from the mobile device at a maximal rate with no on-device filtering or post processing. In some embodiments, the raw IMU data may be subjected to a series of processing. For example, the raw IMU data may be processed using one or more of the following techniques: low pass filtering, high pass filtering, bandpass filtering, savgol filtering, standard deviation, or empirical mode decomposition. The filtering may also involve multiple steps as in the case of a filter bank or multiple stages of different filter types. From the filtered or raw IMU data, further features can be obtained, such as a signal power for a combination of axes. One or more features may be evaluated using a variety of metrics including, e.g., a principal component analysis, recursive feature elimination, LASSO regression, and correlation.

At block 430, the process 400 includes determining the applied force based on a force model and the IMU data.

In some embodiments, the process 400 may include decomposing the IMU data based on an empirical mode decomposition (EMD) technique. According to the EMD technique, the process 400 may include for each axis of the multi-axis accelerometer data and the multi-axis gyroscope data, decomposing a signal of the axis into a plurality of intrinsic mode functions (IMFs) representing different frequency components of the signal, so that the applied force may be determined based on at least a portion of the IMFs that correspond to various axes of the multi-axis accelerometer data and the multi-axis gyroscope data.

Given that the high frequency components of the signals of various axes from the vibration motor are the strongest components of the corresponding signals, the first IMFs of various axes of the IMU data primarily correspond to the vibration motor signal. The upper envelope of the first IMF of each axis of the multi-axis accelerometer data and the multi-axis gyroscope data may be used as a feature for identifying the applied force. In some embodiments, the upper envelope of the first IMF on each IMU axis serves as a representative feature, and the first IMFs collectively may most significantly correlate to the force data and therefore used as input to a force model to determine the applied force, while other features may be excluded. Accordingly, merely by way of example, the process 400 may include for each axis of the multi-axis accelerometer data and the multi-axis gyroscope data, identifying, from the plurality of IMFs of the signal of the axis, a first IMF that corresponds to a highest frequency; and identifying an upper envelope of the first IMF of the axis; and inputting into the force model the upper envelopes of the first IMFs that respectively correspond to all axes of the multi-axis accelerometer data and the multi-axis gyroscope data.

The EMD technique for processing the IMU data is described here for illustration purposes and not intended to be limiting. As described elsewhere in the present document, before being input into the force model, the IMU data may be processed by at least one of filtering, a filter bank, averaging, standard deviation, wavelet decomposition, spectral analysis, or EMD, etc. In some embodiments, signals of at least two different axes of the IMU data (including the multi-axis accelerometer data and the multi-axis gyroscope data) may be processed using different techniques. In some embodiments, the raw IMU data may be directly input into the force model.

The force model may include a machine learning model trained to correlate (1) IMU data measured by the mobile device that is indicative of vibration damping of the mobile device with (2) forces that are applied to the mobile device and cause the corresponding vibration damping. The force model may be a data driven model including parametric modeling, a linear regression model, an ensemble learning model (random forest, adaboost, etc.), a support vector machines model, a neural network (e.g., a transformer model, a convolutional neural network (CNN), etc.), or the like, or a variation thereof, or a combination thereof. Merely by way of example, the force model may include a multivariate linear regression model that provides a value of the applied force on a continuous scale.

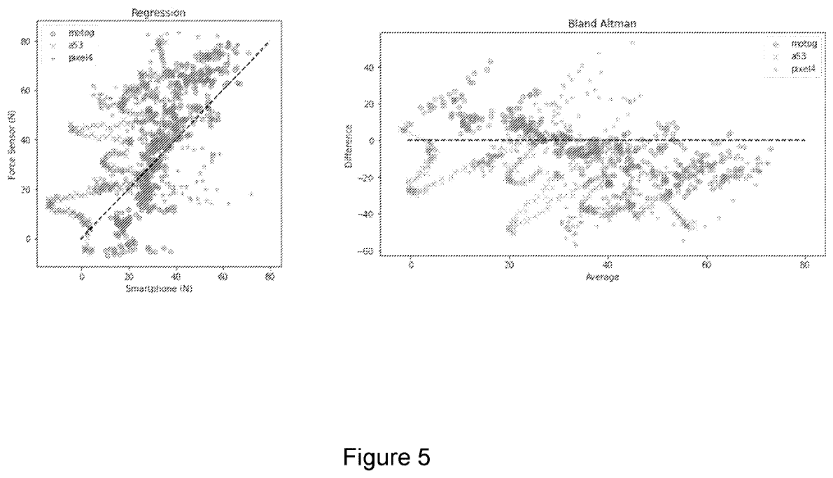

The force model may be trained using training data including applied forces measured using force sensors (e.g., force sensitive resistor (FSR)) and measured dampings caused by the applied forces. An example test case was performed involving three smartphones of different types including Google Pixel 4, Samsung Galaxy A53, and Motorola Moto G Power from a variety of smartphone manufacturers, physical shapes, costs, and embedded components. In the example test case, each participant applied pressure by gripping the smartphone with 0.3 mm thick force sensors positioned under each finger on the side of the smartphone. During the measurement, real-time readings from the force sensors were displayed on a screen in front of the user with a force guide. The user was instructed to use the real time feedback to follow the force guide in applying a range of pressures. The IMU data acquired were processed substantially the same way as described above to provide vibration damping data. The forces measured using the force sensors and the corresponding vibration damping data were used to train and validate the force model. The mean absolute error, correlation coefficient, and bias of each smartphone is displayed in FIG. 5. In FIG. 5, solid dots represent data corresponding to Motorola Moto G Power, crosses represent data corresponding to Samsung Galaxy A53, and stars represent data corresponding to Google Pixel 4.

As discussed elsewhere in the present document, the vibration damping behavior of a mobile device may depend on its configuration. The results from the example test case suggest that relevant differences in force determination by mobile devices of different types (e.g., a type may correspond to a model by a manufacturer) may be calibrated using different force models. A force model may be trained when a mobile device of a new type (e.g., a new model by a manufacture) is released and the obtained force model may be used to calibrate mobile devices of the same type. The type of a mobile device my correspond to a model and/or a manufacturer of the mobile device and associated with factors including, e.g., a configuration of the vibration motor, a configuration of the IMU, or the like, or a combination thereof, of the mobile device.

A model library maya be built by collecting such force models corresponding to mobile devices of different types. In some embodiments, the process 400 may include obtaining, from such a model library, the force model based on a type of the mobile device as a preparation of a hand grip strength measurement procedure.

The process 400 may determine the applied force substantially real time. In some embodiments, during the measurement, the process 400 may include providing a visual guide to be presented on a display of the mobile device, in which the visual guide includes a force plot that illustrates the applied force in substantially real time. In some embodiments, the visual guide may further include a force guide line overlaid with the applied force. The user may be instructed to use the real time feedback to follow the force guide in applying a range of forces.

In some embodiments, the process 400 may include measuring an area of a surface of the body part, in which the force is applied by pressing the surface of the body part against the camera. Merely by way of example, the process 400 may cause the camera to take a picture of the surface of the body part when the body part is pressed against the camera to apply the damping force, and determine the area based on the image. As another example, the mobile device includes a touch screen configured to sense contact by the user, and the process 400 may determine the area by identifying a contact area of the body part with the display. The process 400 may include determining an applied pressure based on the applied force and the area.

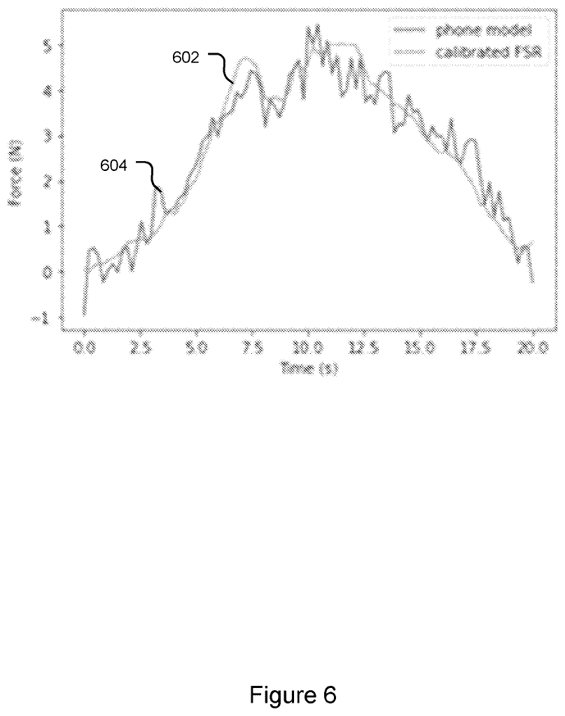

FIG. 6 illustrates a comparison between a force measured using a force sensor (curve 602) with a corresponding force determined based on a force model (curve 604) in accordance with some embodiments of the present document. A calibrated linear force sensitive resistor (FSR) was used to measure the applied force during a grip. As an initial set up, the FSR was placed between a finger of a user and the smartphone. The accelerometer and gyroscope were sampled during a grip while the FSR recorded the applied force. A linear regression fit demonstrates that the smartphone-based HGS measurement may be performed to continuously track grip strength.

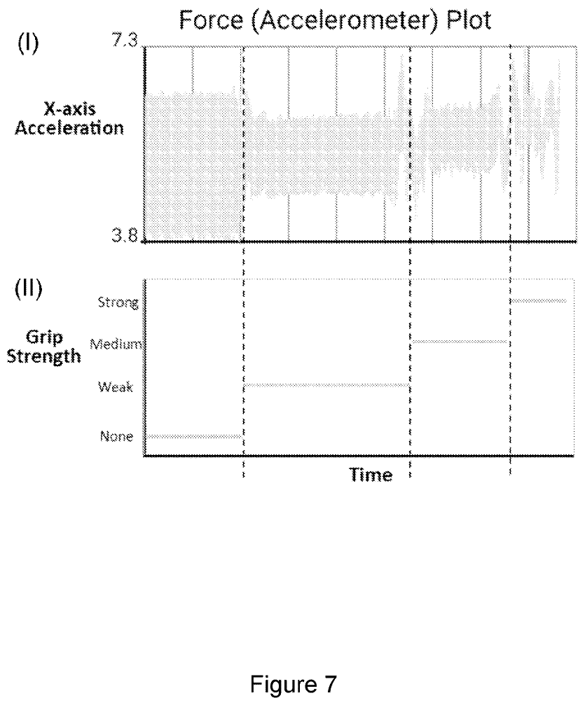

FIG. 7 illustrates results of an HGS measurement using a mobile device categorized as levels in accordance with some embodiments of the present document. Panel (I) illustrates the linear X-axis acceleration of a smartphone IMU over a time period during which the smartphone was vibration and a force was applied to cause vibration damping. Panel (II) illustrates HGS determined according to embodiments of the present disclosure and categorized as levels based on the HGS and/or demographic information of the user. Example relevant demographic information includes age, gender, health status, medical history, or the like, or a combination thereof. As illustrated, higher HGS levels generally correspond to lower linear acceleration along the X-axis.

As described elsewhere in the present document, the IMU of a mobile device may include an accelerometer and a gyroscope. Although only the linear acceleration along the X-axis is illustrated, according to some embodiments of the present document, the IMU of a mobile device measures acceleration in multiple directions (e.g., three perpendicular directions including the direction along the Z-axis and the two directions in a plane perpendicular to the Z-axis), and also gyroscope data in multiple directions (e.g., the same three directions as the accelerometer. The vibration, and a damping thereof, of the mobile device may be monitored based on the IMU data including the multi-axis accelerometer data in combination with the multi-axis gyroscope data. Due to the way vibration damping occurs, the linear accelerations of the IMU in various axes (indicative of the smartphone motion) may change in different patterns. For example, while the applied force increases, the linear acceleration along the X-axis decreases; however, the linear acceleration along a different axis may decrease at a different rate or even increase in some portion(s) of the period the damping force is applied. Similarly, the angular velocities of the IMU along different directions (indicative of the smartphone motion) may change in different patterns in response to the damping force the user applies. Accordingly, the IMU data including multi-axis accelerometer in combination with the multi-axis gyroscope data may be used in determining the applied force.

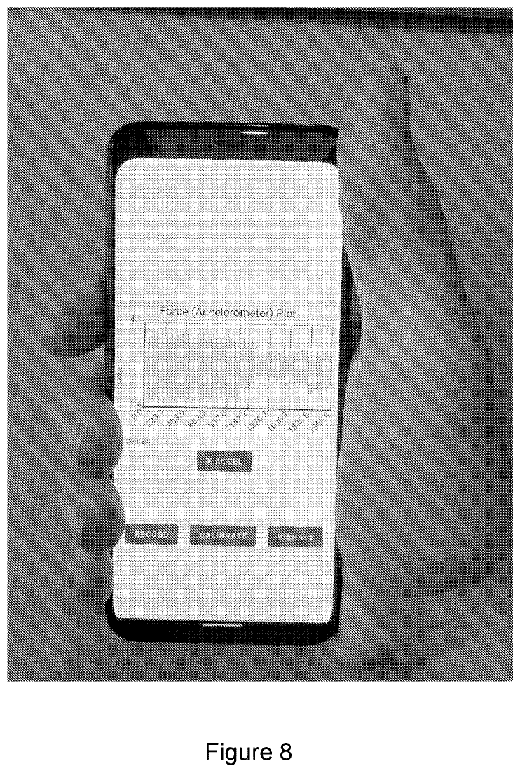

FIG. 8 illustrates an example graphic user interface (GUI) of an application for HGS measurement in accordance with some embodiments of the present document. During the measurement, a user may grip a mobile device (e.g., a smartphone) with a force (e.g., maximum force of the user) for a time period (e.g., 5 seconds), while the mobile device vibrates. The resulting signal can be processed to determine the grip strength in substantially real time. The results may be presented on the GUI real time as illustrated. The GUI may present additional information including, e.g., instructions to guide the user to perform an HGS measurement using the smartphone, features determined based on the determined HGS values, further analysis or actions the user would like (e.g., a different way to present the results (e.g., categorized results, results in text, a summary of the results and relevant information), or a portion thereof, sending the results to another person or device, etc.).

The disclosed mobile device based HGS measure may be used to assess conditions of users. For example, for surgical patients, hand grip strength generally declines following a surgery and recovers as the patient recovers. If the hand grip strength does not recover towards presurgical levels, it may be an indication of surgical complications. Similarly, hand grip strength can be monitored at-home to screen for unexpected drops in strength, which can be indicative of sickness or other states of fragility. As another example, the disclosed technology may be used to assess finger strength for physical exercise readiness. A maximal effort squeeze may be able to assess the body's readiness to perform physical activities of high levels. These squeezes may not necessarily be hand grip strength squeezes. Squeezing the smartphone between the thumb and the forefinger for example may be more effective, especially for individuals with greater strength. A notification relating to the results of HGS measurement of a user may be generated and provided to the user or another person (e.g., a healthcare provider), another device, and/or another entity (e.g., a clinic, a research institute, a device manufacturer). The notification may include a report including the results and/or a condition of the user determined based on the results.

### Examples

The following examples are illustrative of several embodiments in accordance with the present technology. Other exemplary embodiments of the present technology may be presented prior to the following listed examples, or after the following listed examples.
1. A mobile device, including: a vibration motor, an inertial motion unit (IMU), a camera, a processor, and non-transitory computer-readable memory that stores instructions that, when executed by the processor, cause the processor to perform operations including: causing the vibration motor to vibrate; acquiring, using the inertial measurement unit of the mobile device, IMU data indicative of vibration damping that is caused by a user applying a force on the mobile device; and determining the applied force based on a force model and the IMU data.
2. The mobile device of any one or more of the solutions described herein, in which the force model includes a machine learning algorithm trained to correlate (1) the IMU data measured by the mobile device that is indicative of the vibration damping of the mobile device with (2) the force that is applied to the mobile device and cause the vibration damping.
3. The mobile device of any one or more of the solutions described herein, in which the IMU includes an accelerometer and a gyroscope, and the IMU data includes multi-axis accelerometer data and multi-axis gyroscope data.
4. A method for measuring hand grip strength using a mobile device, the method including: causing a vibration motor of the mobile device to vibrate; acquiring, using an inertial measurement unit (IMU) of the mobile device, IMU data indicative of vibration damping that is caused by a user applying a force on the mobile device; and determining the applied force based on a force model and the IMU data.
5. The method of any one or more of the solutions described herein, in which the IMU data includes multi-axis accelerometer data and multi-axis gyroscope data.
6. The method of any one or more of the solutions described herein, in which determining the applied force from the IMU data includes processing the IMU data based on at least one of filtering, a filter bank, averaging, standard deviation, wavelet decomposition, spectral analysis, or EMD.
7. The method of any one or more of the solutions described herein, in which: each axis of the multi-axis accelerometer data and the multi-axis gyroscope data includes a signal, determining the applied force from the IMU data includes for each axis of the multi-axis accelerometer data and the multi-axis gyroscope data, decomposing a signal of the axis into a plurality of intrinsic mode functions (IMFs) representing different frequency components of the signal, and the applied force is determined based on at least a portion of the IMFs that correspond to various axes of the multi-axis accelerometer data and the multi-axis gyroscope data.
8. The method of any one or more of the solutions described herein, in which determining the applied force from the IMU data further includes: for each axis of the multi-axis accelerometer data and the multi-axis gyroscope data, identifying, from the plurality of IMFs of the signal of the axis, a first IMF that corresponds to a highest frequency; and identifying an upper envelope of the first IMF of the axis; and inputting into the force model the upper envelopes of the first IMFs that respectively correspond to all axes of the multi-axis accelerometer data and the multi-axis gyroscope data.
9. The method of any one or more of the solutions described herein, further including: providing a visual guide to be presented on a display of the mobile device, in which the visual guide includes a force plot that illustrates the applied force in substantially real time.
10. The method of any one or more of the solutions described herein, in which the visual guide further includes a force guide line overlaid with the applied force.
11. The method of any one or more of the solutions described herein, further including: providing a visual guide to be presented on a display of the mobile device, in which the visual guide includes indicia to guide the user in applying the force over a time period.
12. The method of any one or more of the solutions described herein, further including: providing a visual guide to be presented on a display of the mobile device, in which the visual guide includes indicia to guide the user in applying the force by gripping the mobile device using a hand of the user.
13. The method of any one or more of the solutions described herein, further including: assessing a condition of the user based on the applied force; and generating a notification based on a result of the assessment.
14. The method of any one or more of the solutions described herein, further including obtaining, from a model library, the force model based on a type of the mobile device, in which the type of the mobile device is associated with at least one of a configuration of the IMU or a configuration of the vibration motor.
15. The method of any one or more of the solutions described herein, in which: the force model includes a machine learning algorithm trained to correlate (1) the IMU data measured by the mobile device that is indicative of the vibration damping of the mobile device with (2) the force that is applied to the mobile device and cause the vibration damping.
16. The method of any one or more of the solutions described herein, in which the force model includes a multivariate linear regression model trained based on training data acquired using a force sensor and a training mobile device of a same type as the mobile device.
17. The method of any one or more of the solutions described herein, in which the force model is configured to generate results on a continuous scale, and the method further includes: assigning the applied force to one of multiple categories.
18. One or more non-transitory computer-readable media storing instructions that, when executed by one or more processors of a mobile device, cause the mobile device to perform any one or more of the solutions described herein.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them.

The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Various embodiments described herein are described in the general context of methods or processes, which may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), Blu-ray Discs, etc. Therefore, the computer-readable media described in the present application include non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

For example, one aspect of the disclosed embodiments relates to a computer program product that is embodied on a non-transitory computer readable medium. The computer program product includes program code for carrying out any one or and/or all of the operations of the disclosed embodiments.

Only a few implementations and examples are described and other implementations, enhancements and variations can be made based on what is described and illustrated in this patent document.

What is claimed is:

1. A method for force measurement using a mobile device, the method comprising:
causing a vibration motor of the mobile device to vibrate;
acquiring, using an inertial measurement unit (IMU) of the mobile device, IMU data indicative of vibration damping that is caused by a user applying a force on the mobile device; and
determining the applied force based on a force model and the IMU data.

2. The method of claim 1, wherein the IMU data comprises multi-axis accelerometer data and multi-axis gyroscope data.

3. The method of claim 2, wherein determining the applied force comprises processing the IMU data based on at least one of filtering, a filter bank, averaging, standard deviation, wavelet decomposition, spectral analysis, or empirical mode decomposition (EMD).

4. The method of claim 3, wherein:
each axis of the multi-axis accelerometer data and the multi-axis gyroscope data comprises a signal,
determining the applied force comprises for each axis of the multi-axis accelerometer data and the multi-axis gyroscope data, decomposing a signal of the axis into a plurality of intrinsic mode functions (IMFs) representing different frequency components of the signal, and
the applied force is determined based on at least a portion of the IMFs that correspond to various axes of the multi-axis accelerometer data and the multi-axis gyroscope data.

5. The method of claim 4, wherein determining the applied force further comprises:
for each axis of the multi-axis accelerometer data and the multi-axis gyroscope data,
identifying, from the plurality of IMFs of the signal of the axis, a first IMF that corresponds to a highest frequency;
identifying an upper envelope of the first IMF of the axis; and
inputting into the force model the upper envelopes of the first IMFs that respectively correspond to all axes of the multi-axis accelerometer data and the multi-axis gyroscope data.

6. The method of claim 1, further comprising:
providing a visual guide to be presented on a display of the mobile device, wherein the visual guide comprises a force plot that illustrates the applied force in substantially real time.

7. The method of claim 6, wherein the visual guide further comprises a force guide line overlaid with the applied force.

8. The method of claim 7, wherein the visual guide comprises indicia to guide the user in applying the force over a time period.

9. The method of claim 1, further comprising:
providing a visual guide to be presented on a display of the mobile device, wherein the visual guide comprises indicia to guide the user in applying the force by gripping the mobile device using a hand of the user.

10. The method of claim 1, further comprising:
assessing a condition of the user based on the applied force; and
generating a notification based on a result of the assessment.

11. The method of claim 1, further comprising:
obtaining, from a model library, the force model based on a type of the mobile device, wherein the type of the mobile device is associated with at least one of a configuration of the IMU or a configuration of the vibration motor.

12. The method of claim 1, wherein the force model comprises a machine learning algorithm trained to correlate (1) the IMU data measured by the mobile device that is indicative of the vibration damping of the mobile device with (2) the force that is applied to the mobile device and cause the vibration damping.

13. The method of claim 1, wherein the force model comprises a multivariate linear regression model trained based on training data acquired using a force sensor and a training mobile device of a same type as the mobile device.

14. The method of claim 1, wherein:
the force model is configured to generate results on a continuous scale, and
the method further comprises: assigning the applied force to one of multiple categories.

15. A mobile device, comprising: a vibration motor, an inertial motion unit (IMU), a processor, and non-transitory computer-readable memory that stores instructions that, when executed by the processor, cause the processor to perform operations including:
causing the vibration motor to vibrate;
acquiring, using the IMU of the mobile device, IMU data indicative of vibration damping that is caused by a user applying a force on the mobile device; and
determining the applied force based on a force model and the IMU data.

16. The mobile device of claim 15, wherein the force model comprises a machine learning algorithm trained to correlate (1) the IMU data measured by the mobile device that is indicative of the vibration damping of the mobile device with (2) the force that is applied to the mobile device and cause the vibration damping.

17. The mobile device of claim 15, wherein the IMU comprises an accelerometer and a gyroscope, and the IMU data comprises multi-axis accelerometer data and multi-axis gyroscope data.

18. One or more non-transitory computer-readable media storing instructions that, when executed by one or more processors of a mobile device, cause the mobile device to perform operations including:
causing a vibration motor of the mobile device to vibrate;
acquiring, using an inertial measurement unit (IMU) of the mobile device, IMU data indicative of vibration damping that is caused by a user applying a force on the mobile device; and
determining the applied force based on a force model and the IMU data.

19. The one or more non-transitory computer-readable media of claim 18, wherein the operations further include:
obtaining, from a model library, the force model based on a type of the mobile device, wherein the type of the mobile device is associated with at least one of a configuration of the IMU or a configuration of the vibration motor.

20. The one or more non-transitory computer-readable media of claim 18, wherein the force model comprises a machine learning algorithm trained to correlate (1) the IMU data acquired from concurrent measurements by an accelerometer and a gyroscope of the mobile device and indicative of the vibration damping of the mobile device with (2) the force that is applied to the mobile device and cause the vibration damping.
